(19) United States
(12) Patent Application Publication
(10) Pub. No.: US 2006/0166224 A1
(43) Pub. Date: Jul. 27, 2006

(54) Associations using genotypes and phenotypes

(76) Inventor: Vernon A. Norviel, San Jose, CA (US)

Correspondence Address: WILSON SONSINI GOODRICH & ROSATI, 650 PAGE MILL ROAD, PALO ALTO, CA, 94304-1050, US

(21) Appl. No.: 11/043,689

(22) Filed: Jan. 24, 2005

(51) Int. Cl. C12Q 1/68; G06F 19/00

(52) U.S. Cl. 435/6; 702/20

(57) ABSTRACT

The present invention discloses methods for combining data on genetic variations and phenotypes of individuals to predict a phenotype-of-interest. The present invention also discloses kits that can be used to determine if an individual has or does not have a phenotype-of-interest. The kit can include at least one diagnostic tool and written instructions.

### BACKGROUND

The DNA that makes up human chromosomes provides the instructions that direct the production of all proteins in the body. These proteins carry out vital functions of life. Variations in DNA are directly related to almost all human diseases, including infectious diseases, cancers, inherited disorders, and autoimmune disorders. Variations in DNA contributing to a phenotypic change, such as a disease or a disorder, may result from a single variation that disrupts the complex interactions of several genes or from any number of mutations within a single gene. For example, Type I and II diabetes have been linked to multiple genes, each with its own pattern of mutations. In contrast, cystic fibrosis can be caused by any one of over 300 different mutations in a single gene. Phenotypic changes may also result from variations in non-coding regions of the genome. For example, a single nucleotide variation in a regulatory region can upregulate or downregulate gene expression or alter gene activity.

Technological developments in the field of human genomics have enabled the development of pharmacogenomics, the use of human DNA sequence variability in the development and prescription of drugs. Pharmacogenomics is based on the correlation or association between a given genotype and a resulting phenotype. Since the first association study over half-a-century ago linking adverse drug response with amino acid variations in two drug-metabolizing enzymes (plasma cholinesterase and glucose-6-phosphate dehydrogenase), other correlation studies have linked sequence polymorphisms in drug metabolism enzymes, drug targets and drug transporters with compromised levels of drug efficacy or safety.

Pharmacogenomics information is especially useful in clinical settings where association information is used to prevent drug toxicities. For example, patients may be screened for genetic differences in the thiopurine methyltransferase gene that cause decreased metabolism of 6-mercaptopurine or azathiopurine. However, only a small percentage of observed drug toxicities have been explained adequately by the set of pharmacogenomic markers available to date. In addition, “outlier” individuals, or individuals experiencing unanticipated effects in clinical trials (when administered drugs that have previously been demonstrated to be both safe and efficacious), cause substantial delays in obtaining FDA drug approval and may even cause certain drugs to come off market, although such drugs may be efficacious for a majority of recipients. Thus, there remains a need for improved methods for predicting phenotypes-of-interest, such as drug response or adverse reactions.

### BRIEF SUMMARY OF THE INVENTION

According to one embodiment, a method is disclosed that includes the steps of identifying one or more genetic variations that at least partly differentiate between individuals with a phenotype-of-interest and individuals without said phenotype-of-interest; identifying one or more phenotypes that at least partly differentiate between said individuals with said phenotype-of-interest and said individuals without said phenotype-of-interest; and predicting based upon said one or more genetic variations and said one or more phenotypes, whether an individual has, does not have, or is at risk of developing said phenotype-of-interest.

### BRIEF DESCRIPTION OF THE DRAWINGS

FIG. 1 is a flow chart illustrating aspects of the method herein.

### DETAILED DESCRIPTION

As used in the specification, “a” or “an” means one or more. As used in the claims, when used in conjunction with the word “comprising”, the words “a” or “an” mean one or more. As used herein, “another” means at least a second or more. As used herein, “individual” means any organism whether prokaryotic or eukaryotic, but preferably a plant or an animal, or more preferably a human.

Reference now will be made in detail to various embodiments and particular applications of the invention. While the invention will be described in conjunction with the various embodiments and applications, it will be understood that such embodiments and applications are not intended to limit the invention. On the contrary, the invention is intended to cover alternatives, modifications and equivalents that may be included within the spirit and scope of the invention. In addition, throughout this disclosure various patents, patent applications, websites and publications are referenced. Unless otherwise indicated, each is incorporated by reference in its entirety for all purposes.

Processes that may be used in specific embodiments of the methods herein are described in more detail in the following patent applications, all of which are specifically incorporated herein by reference: U.S. Provisional Application Ser. No. 60/280,530, and Uses Thereof”; U.S. Provisional Application Ser. No. 60/313,264 filed Aug. 17, 2001, entitled “Identifying Human SNP Haplotypes, Informative SNPs and Uses Thereof”; U.S. Provisional Application Ser. No. 60/327,006, filed Oct. 5, 2001, entitled “Identifying Human SNP Haplotypes, Informative SNPs and Uses Thereof”; U.S. Provisional Application Ser. No 60/332,550, filed Nov. 26, 2002, entitled “Methods for Genomic Analysis”; U.S. application Ser. No. 10/106,097, filed Mar. 26, 2002, entitled “Methods for Genomic Analysis”; U.S. application Ser. No. 10/042,819, filed Jan. 7, 2002, entitled “Genetic Analysis Systems and Methods”; and U.S. application Ser. No. 10/284,444, filed Oct. 31, 2002, entitled “Human Genomic Polymorphisms”, the disclosures all of which are specifically incorporated herein by reference.

All publications mentioned herein are cited for the purpose of describing and disclosing reagents, methodologies and concepts with the present invention. Nothing herein is to be construed as an admission that these references are prior art in relation to the inventions described herein.

Sequencing the human genome has revealed that there is a high degree of homology in genetic information between individuals. In particular, any two humans share approximately 99.9% the same DNA sequence and have up to about 20,000 to about 30,000 or so genes similarly situated in one of twenty-three chromosomes. However, genomic variations between any two individuals still exist. For example, approximately 0.1%, or one out of every 1,000 DNA letters, is different between any two humans.

Genetic variations between individuals can occur in many forms. Examples of genetic variations include, but are not limited to, deletions or insertions of one or more nucleic acids, variations in the number of repetitive DNA elements, and changes in a single nitrogenous base position, also known as “single nucleotide polymorphisms” or “SNPs”. It is noted that any of the genetic variations herein can appear in DNA as well as RNA.

In scanning the human genome, it is estimated that there are 3-4 million common SNPs. Typically, SNPs are biallelic, which means that they occur in two forms, a major allele and a minor allele, with the major allele being more frequently observed than the minor allele. Typically, the major allele occurs in more than 50% of the population; while the minor allele occurs in less than 50% of the population. Common SNPs are those SNPs that have a minor allele frequency of at least about 10%, meaning that the minor allele is present in at least about 10% of individuals. Furthermore, common SNPs do not occur independently but are inherited together from generation to generation in genetic disequilibrium with other SNPs, forming patterns across genomic DNA and RNA. Groups of SNPs that are in linkage disequilibrium with one another define genomic regions that are referred to herein as haplotype blocks. A haplotype block is further characterized by one or more haplotype patterns. A haplotype pattern is the set of SNP alleles on a single nucleic acid strand within a single haplotype block (e.g., on a single chromosome of a single individual). SNP alleles, haplotype patterns, and allelic variations that do not occur in at least about 10% of a given population can be described as rare. Therefore, SNPs with a minor allele frequency of less than about 10% may be referred to herein as “rare SNPs”, and haplotype patterns and allelic variations that occur in less than 10% of the population may be referred to herein “rare haplotype patterns” and “rare allelic variations,” respectively.

Table 1 below illustrates nucleotide bases in six positions from three individuals. The nucleotide base positions can be in genomic DNA or RNA.

TABLE 1

Nucl. Position:

| | 1 | 2 | 3 | 4 | 5 | 6 |
|---|---|---|---|---|---|---|
| Individual 1: | T | A | G | T | C | G |
| Individual 2: | T | A | A | T | C | C |
| Individual 3: | T | A | G | T | C | G |

At nucleotide positions 1-2 and 4-5, all three individuals have the same nucleotide bases. At nucleotide positions 3 and 6, individual 2 has SNP alleles represented by underlined nucleotide bases A and C, respectively, as compared with individuals 1 and 3 who have SNP alleles G and G at the same nucleotide positions.

If both major and minor alleles of SNPs found at positions 3 and 6 above occur in more than about 10% of the population (e.g., major and minor SNP alleles occur at a ratio of 90% and 10%, or 70% and 30%, but not 95% and 5%, respectively), then such SNPs are referred to as common SNPs. Furthermore, if the two SNP alleles (e.g., A and C) at positions 3 and 6 consistently appear together (i.e., are in linkage disequilibrium with one another), then they are part of a haplotype pattern. A haplotype pattern refers to genotyped SNP alleles that consistently appear together. The SNP locations of the SNP alleles in a haplotype pattern form a haplotype block. Haplotype blocks can include known as well as currently unknown SNPs. A SNP whose genotype is predictive of a genotype of one or more other SNPs in a haplotype block are often referred to as “informative SNPs”. For purposes of conducting association studies to predict a phenotype-of-interest, it may be sufficient to scan only one, only two, or only a few informative SNPs from one or more haplotype blocks.

In some embodiments, the present invention contemplates scanning an initial set of nucleotide bases from a plurality of individuals to identify one or more genetic variations (e.g., common SNPs). Such scanning step can occur prior to, contemporaneous with, or after receiving data on the set of phenotypes for such individuals that are selected for an association study. This initial set of bases can come from the same and/or different individuals as those selected for the association study.

Methods for identifying genetic variations are known in the art. For example, the identity of SNPs and SNP haplotype blocks across one representative chromosome (e.g., Chromosome 21) are disclosed in U.S. Provisional Ser. No., 60/323,059, filed Sep. 18, 2001, entitled “Human Genomic Polymorphisms” assigned to the assignee of the present invention; and U.S. application Ser. No. 10/284,444, filed Sep. 18, 2001, entitled “Human Genomic Polymorphisms”, incorporated herein by reference for all purposes. See also Patil, N. et al., “Blocks of Limited Haplotype Diversity Revealed by High-Resolution Scanning of Human Chromosome 21” Science 294, 1719-1723 (2001), disclosing SNPs and haplotype structure of Chromosome 21.

In some embodiments, whole genome analysis is performed to identify genetic variations across the entire genome (DNA and/or RNA). Methods for whole genome analysis can be used both to identify known and/or new variations. Such methods are described in U.S. Provisional Application No. 60/327,006, filed Oct. 5, 2001, entitled “Identifying Human SNP Haplotypes, Informative SNPs and Uses Thereof,” and U.S. application Ser. No. 10/106,097 “Methods For Genomic Analysis”, both of which are assigned to the assignee of the present invention; and U.S. Publication No. 2003/0044780, all of which are incorporated herein by reference for all purposes.

Briefly, in order to scan full genomes, full sets of chromosomes may be separated from samples from individuals (e.g., more than 10, more than 20, more than 30, more than 40, or most preferably more than 50 individuals). This results in multiple unique genomes. Preferably, haploid genomes (or genomes derived from a single set of chromosomes) are used.

In some embodiments, RNA (e.g. MRNA) may be scanned to identify genetic variations. In order to scan RNA, RNA is first isolated from a cell, group of cells, or individuals. Methods for isolating RNA are known in the art. RNA can be isolated from more than 10, more than 20, more than 30, more than 40, or more than 50 individuals. Differences in expression patterns and/or genetic variations in RNA can be identified using any means known in the art or disclosed herein. See e.g. U.S. application Ser. Nos. 10/438,184 and 10/845,316, and PCT/US/04/010699, which are incorporated herein by reference for all purposes.

In some embodiments, all or a significant portion of an individual's genetic material (e.g., DNA, RNA, MRNA, CDNA, other nucleotide bases or derivative thereof) is scanned or sequenced using, e.g., conventional DNA sequencers or chip-based technologies to identify a set of SNPs and their corresponding alleles. In some embodiments, whole-wafer technology from Affymetrix, Inc. of Santa Clara, Calif. is used to read each individual's genome and/or RNA at single-base resolution.

A scanning step (whether to identify new genetic variations or to genotype an individual) can involve scanning at least 10,000 bases, at least 20,000 bases, at least 50,000 bases, at least 100,000 bases, at least 200,000 bases, at least 500,000 bases, at least 1,000,000 bases, more preferably, at least 2,000,000 bases, at least 5,000,000 bases, at least 10,000,000 bases, at least 20,000,000 bases, at least 50,000,000 bases, at least 100,000,000 bases, at least 200,000,000 bases, at least at least 500,000,000 bases, at least 1,000,000,000 bases, at least 2,000,000,000 bases, or at least 3,000,000,000 bases of an individual's genetic material.

In some embodiments, a diagnostic tool that identifies genetic variations scans less than 100,000,000 bases, less than 50,000,000 bases, less than 10,000,000 bases, less than 5,000,000 bases, less than 2,000,000 bases, less than 1,000,000 bases, less than 500,000 bases, less than 200,000 bases, less than 100,000 bases, less than 50,000 bases, less than 20,000 bases, less than 10,000 bases, less than 5,000 bases, less than 2,000 bases, less than 1,000 bases, less than 500 bases, less than 200 bases, less than 100 bases, less than 50 bases, less than 20 bases, or less than 10 bases.

Scanning nucleotide bases in a first set of individuals (e.g., at least 10 individuals, at least 20 individuals, at least 30 individuals, at least 40 individuals, or at least 50 individuals) allows for identification of new genetic variations and/or genetic variations between individuals. Genetic variation data generated from each individual e.g. is compared with genetic variation data generated from other individuals in a first set of individuals in order to discover 1, 2, 3, 4, 5, 6, 7, 8, 9, 10 or more or 10, 20, 30, 40, 50, 60, 70, 80, 90, 100 or more, substantially all or all genetic variations among the first group of individuals.

The variations identified in the first set of individuals can be used in subsequent association studies in which such variations are analyzed to determine if they are associated with a phenotype-of-interest. These variations include, e.g., SNPs, common SNPs, informative SNPs, rare SNPs, deletions, insertions, frameshift mutations, etc. Such genetic variations can be detected in, for example, genomic DNA, RNA, mRNA, or derivatives thereof. In some embodiments, genetic variations scanned and/or identified are informative SNPs. Identification of informative SNPs can reduce the cost and increase the efficiency of association studies because the genotype of a single informative SNP can predict the genotype of one or more other SNP locations.

For example, in conducting whole genome association studies, instead of scanning and reading all 3 billion bases from each genome or about 3 to 4 million common SNPs, it is possible to scan or read simply about 300,000 to 500,000 informative SNPs, which may provide the same amount of information as scanning the entire genome. Thus, while in some embodiments the present invention contemplates scanning whole genomes for association studies, in other embodiments only specific chromosomes, genomic regions, common SNPs, or informative SNPs are scanned and/or used to conduct association studies. Specific chromosomes, genomic regions, common SNPs, or informative SNPs may be selected for association studies based on prior knowledge that such regions are related to a particular phenotype-of-interest (e.g., disease state or lack thereof).

The present invention contemplates association studies using genetic variations and phenotypes of individuals from both case and control groups. Case group individuals are those who express a phenotype-of-interest. Control group individuals are those who do not express a phenotype-of-interest. In some embodiments, a case group includes at least 2, 5, 10, 20, 50, 100, 200, 500, or 1000 individuals and a control group includes at least 2, 5, 10, 20, 50, 100, 200, 500, or 1000 individuals. Methods for performing genotype association studies using case and control groups are described, e.g., in U.S. Ser. No. 10/351,973, filed Jan. 27, 2003, entitled “Apparatus and Methods for Determining Individual Genotypes”; in U.S. Ser. No. 10/786,475, filed Feb. 24, 2004, entitled “Improvements to Analysis Methods for Individual Genotyping”; and in U.S. Ser. No. 10/970,761, filed Oct. 20, 2004, entitled “Improved Analysis Methods and Apparatus for Individual Genotyping”, all of which are incorporated herein by reference for all purposes.

To increase efficiency of collecting genotyping data, cases and/or controls can be pooled prior to scanning as is described in U.S. application Ser. No. 10/447,685, filed May 28, 2003, entitled “Liver Related Disease Compositions and Methods”, U.S. application Ser. No. 10/427,696; filed Apr. 30, 2003; entitled “Methods for Identifying Matched Groups”; and U.S. application Ser. No. 10/768,788; filed Jan. 30, 2004; entitled “Apparatus and Methods for Analyzing and Characterizing Nucleic Acid Sequences” which are incorporated herein by reference. For example, samples obtained from all or some case individuals and/or all or some control individuals may be pooled together prior to scanning. In another example, data on genetic variations and/or phenotypes from some or all case individuals and/or some or all control individuals may be pooled together. Furthermore, in any of the embodiments herein, genetic variation data collected can be stored in a computer readable medium for further analysis.

In any of the embodiments herein, a scanning step (for either identifying or genotyping variations) may be supplemented and/or substituted by receiving data on the genetic variations from database(s). Such databases can provide, for example, a list of identified genetic variations (e.g., SNPs or haplotypes) or genotyping data on particular individuals. Examples of publicly available databases that identify genetic variations include, but are not limited to, NCBI's dbSNP <http://www.ncbi.nlm.nih.gov/SNP/index.html>; MIT's human SNP database <http://www.broad.mit.edu/snp/human/>; University of Geneva's human Chromosome 21 SNP database (http://csnp.unige.ch/>; and the University of Tokyo's SNP database <http://snp.ims.u-tokyo.ac.jp/>. Other databases known in the art may be used in conjunction with the methods herein.

The present invention contemplates the use of genetic variations between individuals (e.g., SNP alleles, and haplotype patterns) along with a set of phenotypes of the individuals in association studies to predict if an individual has or does not have a phenotype-of-interest. Association studies using only genetic variations are described in U.S. application Ser. No. 10/447,685, filed May 28, 2003, entitled “Liver Related Disease Compositions and Methods” which is incorporated herein by reference.

Like genotyping data, data on a set of phenotypes of the individuals is received for both case individuals and control individuals. The data on a set of phenotypes preferably includes data on at least 1, 2, 3, 4, 5, 6, 7, 8, 9, or 10 different phenotypes, or more preferably on at least 10, 25, 30, 35, 40, 45 or 50 different phenotypes of the individuals in the association study. The data on the set of phenotypes can be collected prior to, subsequent to, or simultaneous with the collection/gathering of genotyping data. Phenotype data collected can (like the genotyping data) also be stored in a computer readable medium for further use.

Both the genotyping data and the phenotyping data on the group of individuals is used simultaneously in an association study for a phenotype-of-interest. Results from the association study can be commercialized in any form of e.g., data, kits, and/or improved drugs.

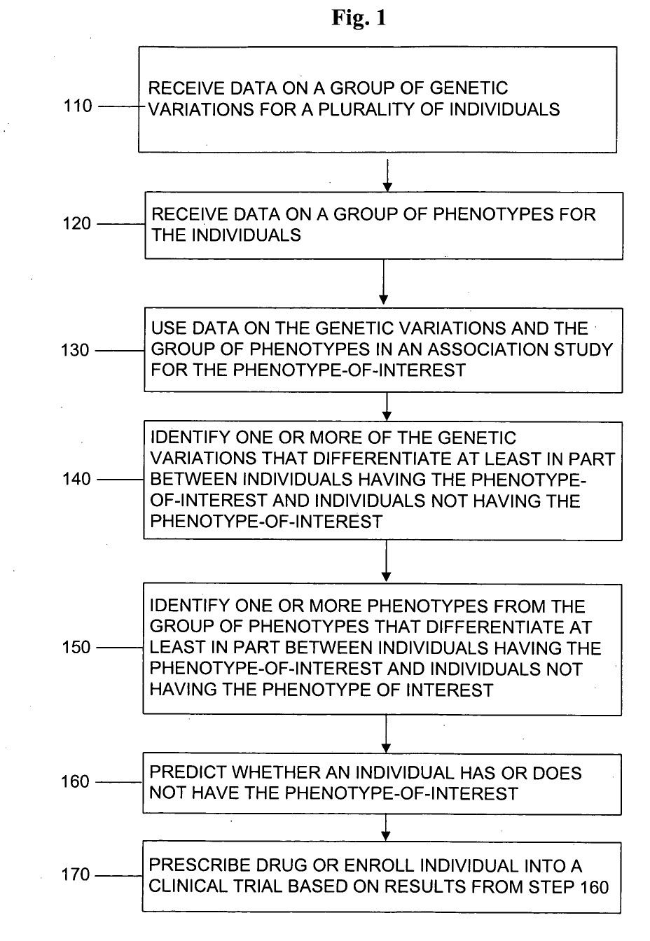

FIG. 1 illustrates one embodiment of the systems and methods herein. At step 110, data on genetic variations from a plurality of individuals with and without a phenotype-of-interest is received. The plurality of individuals preferably includes at least 10, at least 20, at least 30, at least 40, or at least 50 individuals with a phenotype-of-interest and at least 10, at least 20, at least 30, at least 40, or at least 50 individuals without the phenotype-of-interest. In some embodiments data on genetic variations is derived by scanning genetic material (e.g., DNA, RNA, mRNA, cDNA, or derivatives thereof) of the individuals. In other embodiments, such data may be derived from a database.

Scanning for genetic variations can involve scanning of at least 10,000 bases, at least 20,000 bases, at least 50,000bases, at least 100,000 bases, at least 200,000 bases, at least 500,000 bases, at least 1,000,000 bases, at least 2,000,000 bases, at least 5,000,000 bases, at least 10,000,000 bases, at least 20,000,000 bases, at least 50,000,000 bases, at least 100,000,000 bases, at least 200,000,000 bases, at least at least 500,000,000 bases, at least 1,000,000,000 bases, at least 2,000,000,000 bases, or at least 3,000,000,000 bases of genetic material from an individual. In such scanning, genetic variations can be both discovered and genotyped.

In some embodiments a diagnostic tool that identifies genetic variations can scan less than 100,000,000 bases, less than 50,000,000 bases, less than 10,000,000 bases, less than 5,000,000 bases, less than 2,000,000 bases, less than 1,000,000 bases, less than 500,000 bases, less than 200,000 bases, less than 100,000 bases, less than 50,000 bases, less than 20,000 bases, less than 10,000 bases, less than 5,000 bases, less than 2,000 bases, less than 1,000 bases, less than 500 bases, less than 200 bases, less than 100 bases, less than 50 bases, less than 20 bases, or less than 10 bases.

The genetic variations identified can be, e.g., SNPs, common SNPs, or informative SNPs. In some embodiments, the genetic variations identified include rare SNPs. If informative SNPs are genotyped, it is not necessary to genotype all other SNPs in the same haplotype block. In some embodiments, no more than 1, 2, 3, 4, 5, 6, 7, 8, 9, 10, 20, 30, 40, 50, 75, or 100 SNPs per haplotype block are genotyped. Moreover, it is not necessary to use all of the SNP genotypes in an association study. In some embodiments, only a subset of the total genotypes is used in an association study.

In some embodiments, data on one or more, 2 or more, 5 or more, 10 or more, 15 or more, 20 or more, 25 or more, 30 or more, 35 or more, 40 or more, 45 or more, 50 or more, 60 or more, 70 or more, 80 or more, 90 or more, or 100 or more genetic variations for individuals having a phenotype-of-interest (cases) and individuals not having the phenotype-of-interest (controls) is received for an association study.

Examples of phenotypes-of-interest include, but are not limited to, the appearance of a disease (e.g., cancer, inflammation, diabetes, cardiovascular disease, immunological disease), a drug response (whether positive or negative), etc. In preferred embodiments, the phenotype-of-interest is a drug response. More preferably, the phenotype-of-interest is a drug response that would include or exclude an individual from a drug trial or a drug therapy. See U.S. Provisional No. 60/566,302, filed Apr. 28, 2004, entitled “Methods for Genetic Analysis”; U.S. Provisional No. 60/590,534, filed Jul. 22, 2004, entitled “Methods for Genetic Analysis,” and U.S. Ser. No. 10/956,224, filed Sep. 30, 2004, entitled “Methods for Genetic Analysis,” all of which are incorporated herein by reference for all purposes.

At step 120, data on a group of phenotypes of the plurality of individuals are received. The group of phenotypes includes the phenotype-of-interest. Data on the group of phenotypes can be received prior to, after, and/or concurrent with the receipt the data of the genetic variations in step 110. In some embodiments, data on the group of phenotypes is generated by a practitioner of the present invention by, for example, observation (e.g., gross phenotypic trait), biochemical testing (e.g., blood or urine analysis), or other diagnostic test (e.g., X-ray, MRI, CAT scan, CT scan, Doppler shift, etc.).

Examples of phenotype data that may be received/collected include, but are not limited to, data about the individuals': ability to roll the tongue, ability to taste PTC, acute inflammation, adaptive immunity, addiction(s), adipose tissue, adrenal gland, age, aggression, amino acid level, amyloidosis, anogenital distance, antigen presenting cells, auditory system, autonomic nervous system, avoidance learning, axial defects or lack thereof, B cell deficiency, B cells, B lymphocytes (e.g. antigen presentation), basophils, bladder size/shape, blinking, blood chemistry, blood circulation, blood glucose level, blood physiology, blood pressure, body mass index, body weight, bone density, bone marrow formation/structure, bone strength, bone/skeletal physiology, breast size/shape, bursae, cancellous bone, cardiac arrest, cardiac muscle contractility, cardiac output, cardiac stoke volume, cardiomyopathy, cardiovascular system/disease, carpal bone, catalepsy, cell abnormalities, cell death, cell differentiation, cell morphology, cell number, cell-mediated immunity, central nervous system, central nervous system physiology, chemotactic factors, chondrodystrophy, chromosomal instability, chronic inflammation, circadian rhythm, circulatory system, cleft chin, clonal anergy, clonal deletion, T and B cell deficiencies, conditioned emotional response, congenital skeletal deformities, contextual conditioning, cortical bone thickness, craniofacial bones, craniofacial defects, crypts of Lieberkuhn, cued conditioning, cytokines, delayed bone ossification, dendritic cells (e.g. antigen presentation), Di George syndrome, digestive function, digestive system, digit dysmorphology, dimples, discrimination learning, drinking behavior, drug abuse, drug response, ear size/shape including ear lobe attachment, eating behavior, ejaculation function, embryogenesis, embryonic death, embryonic growth/weight/body size, emotional affect, enzyme/coenzyme level, eosinophils, epilepsy, epiphysis, esophagus, excretion physiology, extremities, eye blink conditioning, eye color/shape, eye physiology, eyebrows shape, eyelash length, face shape, facial cleft, femur, fertility/fecundity, fibula, finger length/shape, fluid regulation, fontanels, foregut, fragile skeleton, freckles, gall bladder, gametogenesis, gastrointestinal hemorrhage, germ cells (e.g., morphology, depletion), gland dysmorphology, gland function, glucagon level, glucose homeostasis, glucose tolerance, glycogen catabolism, granulocytes, granulocytes (e.g., bactericidal activity, chemotaxis), grip strength, grooming behavior, hair color, hair follicle structure/orientation, hair growth, hair on mid joints, hair texture, handedness, harderian glands, head, hearing function, heart, heart rate, heartbeat (e.g. rate, irregularity), height, hemarthrosis, hemolymphoid system, hepatic system, hitchhiker's thumb, homeostasis, humerus, humoral immune response, hypoplastic axial skeleton, hypothalamus, immune cell, immune system (e.g., hypersensitivity), immune system response/function, immune tolerance, immunodeficiency, inability to urinate, increased sensitivity to gamma-irradiation, inflammatory mediators, inflammatory response, innate immunity, inner ear, innervation, insulin level, insulin resistance, intestinal bleeding, intestine, ion homeostasis, jaw, kidney hemorrhage, kidney stones, kidney/renal system, kyphoscoliosis, kyphosis, lacrimal glands, larynx, learning/memory, leukocyte, ligaments, limb dysmorphology, limb grasping, lipid chemistry, lipid homeostasis, lips size/shape, liver (e.g. development/function), liver/hepatic system, locomotor activity, lordosis, lung, lung development, lymph organ development, macrophages (e.g. antigen presentation), mammary glands, maternal/paternal behavior, mating patterns, meiosis, mental acuity, mental stability, mental state, metabolism of xenobiotics, metaphysis, middle ear, middle ear bone, morbidity and mortality, motor coordination/balance, motor learning, mouth, movement, muscle, muscle contractility, muscle degeneration, muscle development, muscle physiology, muscle regeneration, muscle spasms, muscle twitching, musculature, myelination, myogenesis, nervous system, neurocranium, neuroendocrine glands, neutrophils, NK cells, nociception, nose, nutrients/absorption, object recognition memory, ocular reflex, odor preference, olfactory system, oogenesis, operant or “target response”, orbit, osteogenesis, osteogenesis/developmental, osteomyelitis, osteoporosis, outer ear, oxygen consumption, palate, pancreas, paralysis, parathyroid glands, pelvis girdle, penile erection function, perinatal death, peripheral nervous system, phalanxes, pharynx, photosensitivity, piloerection, pinna reflex, pituitary gland, PNS glia, postnatal death, postnatal growth/weight/body size, posture, premature death, preneoplasia, propensity to cross the right arm over the left of vice versa, propensity to cross the right thumb over the left thumb when clasping hands or vise versa, pulmonary circulation, pupillary reflex, radius, reflexes, reproductive condition, reproductive system, resistance to fatty liver development, resistance to hyperlipidemia, respiration (e.g., rate, shallowness), respiratory distress or failure, respiratory mucosa, respiratory muscle, respiratory system, response to infection, response to injury, response to new environment (transfer arousal), ribs, salivary glands, scoliosis, sebaceous glands, secondary bone resorption, seizures, self tolerance, senility, sensory capabilities, sensory system physiology/response, sex, sex glands, shoulder, skin, skin color, skin texture/condition, skull, skull abnormalities, sleep pattern, social intelligence, somatic nervous system, spatial learning, sperm count, sperm motility, spermatogenesis, startle reflex, sternum defect, stomach, suture closure, sweat glands, T cell deficiency, T cells (e.g., count), tarsus, taste response, teeth, temperature regulation, temporal memory, tendons, thyroid glands, tibia, touch/nociception, trachea, tremors, trunk curl, tumor incidence, tumorigenesis, ulna, urinary system, urination pattern, urine chemistry, urogenital condition, urogenital system, vasculature, vasoactive mediators, vertebrae, vesicoureteral reflux, vibrissae, vibrissae reflex, viscerocranium, visual system, weakness, widows peak or lack thereof, etc.

Additional examples of phenotype data that may be received/collected about individuals can include phenotype data about previous medical conditions or medical history (e.g., whether an individual has had surgery, experienced a particular illness, given natural or artificial childbirth, been diagnosed with mental illness, has allergies, etc.).

In some embodiments, phenotype data may also be received/collected on the individuals' family history. For example, data can be collected on relatives suffering from or affected by baldness, cancer, diabetes, hypertension, mental illness, mental retardation, attention deficit, infertility, erectile dysfunction, cardiovascular disease, allergies, drug addiction, etc.

Data on one or more phenotypes is received for individuals with a phenotype-of-interest and without the phenotype-of-interest. Preferably, a larger set of possible phenotypes is used in the association study to provide the greatest probability of identifying the phenotype-of-interest in an individual who may or may not be in case or control groups. For example, data on more than 2, more than 3, more than 5, more than 7, more than 10, more than 15, more than 20, more than 25, more than 30, more than 35, more than 40, more than 45, more than 50, more than 60, more than 70, more than 80, more than 90, or more than 100 phenotypes may be used in an association study.

Data on the group of phenotypes may be received in a binary system (e.g., 0's and 1's) or a greater-fold system (e.g., three-fold, four-fold, etc., such as 0's, 1's, 2's, etc.) on a phenotype-by-phenotype basis. An example of phenotypic data that may be received in a binary system includes the presence (or absence) of a disease. If an individual has a particular phenotype (e.g., disease) from a group of phenotypes, that phenotype may be designated as “1”. Conversely, if an individual does not have a particular phenotype from a group of phenotypes, that phenotype may be designated as “0”.

Similarly, data on the group of phenotypes may also be received in a greater-fold system, such as a three-fold, four-fold system, or a greater-fold system (e.g., more than 10-fold, more than 20-fold, or more than 40-fold). In greater-fold systems each of the multiple forms of a phenotype may be designated with a different number. For example, if an individual expresses a first form (e.g., blue eyes) of a phenotype (e.g., eye color) of a group of phenotypes, that phenotype may be designated as “1”, a second form (e.g., green eyes) of the phenotype of a group of phenotypes may be designated as “2”, a third form (e.g., brown eyes) of the phenotype of a group of phenotypes may be designated as “3”, etc.

Data on the plurality of phenotypes about an individual can also include data about a degree to which such phenotypes or plurality of phenotypes is present (or absent) in the individual. For example, the degree of skin pigmentation can be expressed as a gradient from 1 to 10 wherein “1” represents the lightest skin color and “10” represents the darkest skin color. Determination of the degree of skin pigmentation can be made by an observer (e.g., clinician) or can be made based on a plurality of other determinants using various mathematical-statistical methods including, but not limited to, multiple comparison (Bonferroni), variance analysis, regression and correlation analysis, and multivariant discriminant analysis (see U.S. Pat. No. 4,791,998, which is incorporated herein by reference for all purposes).

At step 130, the genetic variations and the data on the group of phenotypes are used collectively in association studies with one (or more) phenotypes-of-interest. Alternatively, or in addition, the correlation may be conducted through pooling samples to reduce overall costs or by genotyping individual samples. Pooling involves, for example, an additional step prior to the scanning step in which individual DNA samples from a plurality of individuals (either cases or controls) are pooled together and then scanned together to identify SNPs that have a significantly different allele frequency in cases versus controls. The SNPs are not separately genotyped in each individual, but a ratio of each allele is identified in the case and control groups. Methods of pooling are disclosed in U.S. application Ser. No. 10/447,685, filed May, 28, 2003, entitled “Liver Related Disease Compositions and Methods”, U.S. application Serial No. 10/427,696; filed Apr. 30, 2003; entitled “Methods for Identifying Matched Groups”; and U.S. application Ser. No. 10/768,788; filed Jan. 30, 2004; entitled “Apparatus and Methods for Analyzing and Characterizing Nucleic Acid Sequences” which are incorporated herein by reference.

At step 140, one or more genetic variations are identified that differentiate at least in part among individuals having and not having the particular phenotype-of-interest(s). This can be achieved by identifying genetic variations with significant allele frequency differences between cases and controls. Examples of methods for identifying genetic variations with significant allele frequency between cases and controls are disclosed in U.S. application Ser. No. 10/768,788, filed on Jan. 30, 2004, entitled “Apparatus and Methods for Analyzing and Characterizing Nucleic Acid Sequences”, which is incorporated herein by reference.

As used herein, the term “differentiate at least in part” means a clinically useful result that can be used to differentiate cases from controls and is preferably at least 50% sensitive, more preferably at least 60% sensitive, more preferably at least 70% sensitive, more preferably at least 80% sensitive, more preferably at least 90% sensitive, more preferably at least 95% sensitive, or more preferably at least 99% sensitive; or a clinically useful result that can be used to differentiate cases from controls and is preferably at least 50% specific, more preferably at least 60% specific, more preferably at least 70% specific, more preferably at least 80% specific, more preferably at least 90% specific, more preferably at least 95% specific, or more preferably at least 99% specific.

At step 150, one or more phenotypes from the group of phenotypes are identified that can differentiate at least in part among individuals having and not having the particular phenotype-of-interest(s). This can be achieved by identifying phenotypes from the group of phenotypes with significant frequency differences between cases and controls. In certain embodiments, steps 140 and 150 occur simultaneously.

At step 160, it is predicted whether an individual (that can be from neither the case nor the control groups) has or does not have a particular phenotype-of-interest. Step 170 is optional. In step 170, a treatment, such as a drug treatment or radiation treatment is administered (or not administered) to a patient, or a patient is enrolled in a clinical trial, based on the results in step 160.

Table 2 below illustrates hypothetical data received from six individuals. The data includes information on four genetic variations (common SNPs) and four phenotypes. For SNPs, the following letter symbols are used: (A) adenine (T) thymine (C) cytosine, and (G) guanine to indicate SNP alleles.

TABLE 2
Association Study Using Common SNPs (CSs) and Phenotypes (Phs)

| | Phenotype- | | | | | | | | |
|---|---|---|---|---|---|---|---|---|---|
| Individual | of-interest | SNP 1 | SNP 2 | SNP 3 | SNP 4 | Phenotype 1 | Phenotype 2 | Phenotype 3 | Phenotype 4 |
| 1 | 1 | A | C | G | T | 1 | 0 | 2 | 7 |
| 2 | 1 | A | T | G | T | 1 | 0 | 1 | 8 |
| 3 | 0 | T | C | C | A | 0 | 1 | 0 | 1 |
| 4 | 0 | T | A | C | A | 0 | 1 | 2 | 2 |
| 5 | 1 | A | T | G | T | 1 | 0 | 2 | 9 |
| 6 | 0 | T | T | C | A | 0 | 1 | 0 | 1 |

As illustrated by Table 2, individuals 1, 2, and 5 have the phenotype-of-interest (symbolized by a “1”) are cases, while individuals 3, 4, and 6 do not have the phenotype-of-interest (symbolized by a “0”) are controls. The presence of “A” allele at SNP 1, a “G” allele at SNP3, and/or a “T” allele at SNP4 are associated with an individual having the phenotype-of-interest (“1”); while the presence of an “T” allele at SNP1, “C” allele at SNP3, and/or an “A” allele at SNP4 is associated with an individual not having the phenotype-of-interest (“0”).

Similarly, a phenotype score of “1” for phenotype 1, a phenotype score of “0” for phenotype 2, and/or a phenotype score of “7 or higher” for phenotype 4 is associated with an individual having a phenotype-of-interest (“1”); while a phenotype score of “0” for phenotype 1, a phenotype score of “1” score for phenotype 2, and/or a phenotype score of “2 or less” is associated with an individual not having a phenotype-of-interest (“0”).

Combining these data into a single association study, one can predict that an individual with an “A” allele at SNP1, “G” allele at SNP3, and/or “T” at SNP4, having a phenotype score of “1” for phenotype 1, phenotype score “0” for phenotypes 2, and/or phenotype score of “7 or higher” for phenotype 4, will have a phenotype-of-interest (“1”) Conversely, an individual with a “T” allele at SNP1, a “C” allele SNP3, and/or an “A” allele at SNP4, having a phenotype score of “0” for phenotype 1, phenotype score of “1” for phenotype 2, and/or phenotype score of “2 or less” for phenotype 4 will not have a phenotype-of-interest (“0”).

The present invention also contemplates kits for predicting if an individual has or does not have a phenotype-of-interest. Such kits can be used, for example, to identify individuals who may benefit (or not benefit) from a therapeutic treatment, individuals who may be enrolled (or excluded) from a clinical trial, individuals who may suffer (or not suffer) an adverse reaction from a therapeutic treatment, and individuals who be susceptible (or resistant) to a condition or disease. The kits herein may also be used to identify and validate drug target regions, evaluate genetic variations and phenotypes that may be related to susceptibility or resistance to disease, identify genetic variations that may be triggered by environmental cues (e.g., radiation, nutrition, etc.), and evaluate of other genotype-phenotype associations with commercial potential, such as in consumer products and agriculture.

The kits herein preferably include at least one diagnostic tool and a set of written instructions. In some embodiments, the diagnostic tool provides means for identifying one or more genetic variations in an individual. Examples of diagnostic tools that can be used to identify genetic variations include, but are not limited to, a primer, a probe, an immunoassay, a chip based DNA assay, a PCR assay, a Taqman™ assay, a sequencing based assay, and the like. In some embodiments, such tools can provide means for detecting 1 or more genetic variations, more preferably 3 or more genetic variations, more preferably 30 or more genetic variations, more preferably 300 or more genetic variations, more preferably 3,000 or more genetic variations, more preferably 30,000 or more genetic variations, more preferably 300,000 or more genetic variations, or more preferably 3,000,000 or more genetic variations. Preferably, such genetic variations are SNPs.

In some embodiments, a diagnostic tool that identifies genetic variations scans at least 10,000 bases, at least 20,000 bases, at least 50,000 bases, at least 100,000 bases, at least 200,000 bases, at least 500,000 bases, at least 1,000,000 bases, more preferably, at least 2,000,000 bases, at least 5,000,000 bases, more preferably at least 10,000,000 bases, at least 20,000,000 bases, at least 50,000,000 bases, at least 100,000,000 bases, at least 200,000,000 bases, at least at least 500,000,000 bases, at least 1,000,000,000 bases, at least 2,000,000,000 bases, or at least 3,000,000,000 bases of genetic material from an individual. In certain embodiments, not all associated SNPs need to be scanned to determine if an individual has or does not have a phenotype-of-interest.

In some embodiments a diagnostic tool that identifies genetic variations scans less than 100,000,000 bases, less than 50,000,000 bases, less than 10,000,000 bases, less than 5,000,000 bases, less than 2,000,000 bases, less than 1,000,000 bases, less than 500,000 bases, less than 200,000 bases, less than 100,000 bases, less than 50,000 bases, less than 20,000 bases, less than 10,000 bases, less than 5,000 bases, less than 2,000 bases, less than 1,000 bases, less than 500 bases, less than 200 bases, less than 100 bases, less than 50 bases, less than 20 bases or less than 10 bases.

In some embodiments, SNPs scanned and genotyped from part or all of the genome are used in an association study. In other embodiments, only a subset of those SNPs scanned are used in an association study.

In some embodiments, a diagnostic tool provides means for detecting and/or quantifying one or more phenotypes in an individual. Examples of such diagnostic tools include, but are not limited to blood tests (e.g., PSA, blood glucose levels, etc.); other biochemical tests (e.g., pregnancy tests, allergy tests, etc.), self-diagnosis tests (e.g., breast exam, skin exam, IQ exam, etc.); and simple measurements (e.g., weight, height, girth, etc.).

In some embodiments, a kit comprises at least two diagnostic tools: one to detect and/or quantify genetic variation(s) in an individual and one to detect and/or quantify phenotypic trait(s) of the individual. In some embodiments, the written instructions provide guidelines for using the results from the diagnostic tools to predict whether an individual has or does not have a phenotype-of-interest.

The results of the association studies and/or kits herein can be used, directly or indirectly, in drug discovery, clinical trials and other discovery efforts with partners. In some embodiments, the present application contemplates computer readable databases comprising data on genetic variations and a group of phenotypes of individuals. The databases can be accessible on-line or by other medium. The databases can be used to perform virtual association studies to correlate phenotypes and genotypes with a phenotype-of-interest. For example, in some embodiments, databases herein can be used to perform virtual association studies by using one of the phenotypes as a phenotype-of-interest in a new study.

For example, the association studies and/or kits herein can be used to predict if an individual will or will not have a phenotype-of-interest, such as a negative (or positive) drug response based on their genotypes at a set of SNPs or subset thereof and a set or subset of phenotypes. In some embodiments, such drug response may be to a drug or product that has been pulled off the market due to unpredictable adverse effects in a small group of individuals or to one that did not obtain regulatory approval due to a large number of individuals experiencing unanticipated effects in clinical trials.

The data and information generated by the assays disclosed is valuable to numerous industries. For example, information concerning potential drug targets is highly valuable to the biotech industry and can greatly speed up the drug discovery process, and hence time-to-market. Similarly, information concerning the characteristics (effectiveness, safety, and efficiency) of a given drug is extremely valuable to the pharmaceutical industry and can save a company substantial money in lost revenue due to failures in clinical trials. The information generated herein may also be valuable to the agricultural industry, veterinary medicine industry, consumer products industry, insurance and healthcare provider industry and forest management (by providing genetic basis for useful traits in plants, trees, laboratory animals and domestic animals), for example.

Thus, in some embodiments, a collaborator or partner (e.g., a drug company) can use the association studies or kits herein to correlate between genomic and phenotype differences, and e.g., drug response (or lack thereof) or drug tolerance. Furthermore, the ability to predict a phenotype-of-interest, such as drug response, can subsequently be used to stratify patients into various groups. The groups may be, for example, those that respond to a drug versus those that do not respond, or those that respond to a drug without toxic effects versus those that are observed to have toxic effects. This may be useful for such company to overcome negative clinical trial results, obtain regulatory approval faster, and recoup losses. This can also save millions of dollars in unsuccessful clinical trials and fruitless research and development efforts.

Thus, in one embodiment, a therapeutic may be marketed with a kit as disclosed herein that is capable of segregating individuals that will respond in an acceptable manner to a drug from those that will not (e.g., individuals who will experience adverse side effects, minimal beneficial effects or no beneficial effects). Additional methods of using an association study for pharmacogenomics are disclosed in e.g., U.S. Provisional No. 60/566,302, filed Apr. 28, 2004, and entitled “Methods of Genetic Analysis”; U.S. Provisional No. 60/590,534, filed Jul. 22, 2004, and entitled “Methods of Genetic Analysis”; U.S. Provisional No. 10/956,224, filed Sep. 30, 2004, and entitled “Methods of Genetic Analysis”, which are incorporated herein by reference for all purposes.

In any of the embodiments herein, the genomic sequences identified as associated with a phenotype-of-interest by the methods of the present invention may be genic or nongenic sequences. The term “gene” as used herein is intended to mean an open reading frame encoding one or more specific RNAs and/or polypeptides; the RNAs and/or polypeptides encoded by such open reading frames; nucleic acids complementary to the open reading frame or to the encoded RNA; derivatives of the open reading frame or encoded RNA; derivatives of the encoded polypeptides; intronic regions generally and adjacent 5′ and 3′ non-coding nucleotide sequences involved in the regulation of expression of the gene up to about 10 kb beyond the coding region but possibly further in either direction. The coding sequences (ORFS) of a gene may affect a phenotypic state e.g., by affecting protein or RNA structure. Alternatively, the non-coding sequences of the gene or nongenic sequences may affect a phenotype state e.g., by impacting the level of expression or specificity of expression of a protein or RNA.

Genomic sequences identified by the methods presented herein may be further studied by isolating the identified genomic sequence such that it is substantially free of other nucleic acid sequences that do not include the identified genomic sequence. The isolated sequences may subsequently be used in a variety of ways. For example, the isolated nucleic acid sequences may be used to design probes and primers to detect or quantify expression of a gene in a biological specimen. The manner in which one probes cells for the presence of particular nucleotide sequences is well established in the literature and does not require elaboration here, see, e.g., Sambrook, et al., Molecular Cloning: A Laboratory Manual (Cold Spring Harbor Laboratory, New York) (1989). Gene and/or gene segments identified in association with a phenotype of interest can be cloned into expression vectors and expressed in host cells. Expression vectors can include those used for gene therapy and those used for expression in prokaryotic cells. Furthermore, the genomic sequences identified can be used to identify novels genes associated with the phenotype-of-interest. Furthermore, by understanding both the genetic and phenotypic bases of disease (or disease resistance), it may be possible to identify new therapeutic and/or diagnostic targets.

According to one aspect of the invention, scanning involves the use of glass wafers on which high-density arrays of nucleic acid probes have been placed. Each of these wafers holds, for example, approximately 60 million nucleic acid probes that can be used to recognize complementary nucleic acid sequences in a sample. The recognition of sample nucleic acids by the set of nucleic acid probes on the glass wafer takes place through the mechanism of hybridization. When a sample nucleic acid hybridizes with an array of nucleic acid probes, the sample will bind to those probes that are complementary to sample nucleic acid sequence. By evaluating the level of hybridization of different probes to the sample nucleic acid, it is possible to determine whether a known sequence of nucleic acid is present or absent in the sample. See, e.g., U.S. Pat. Nos. 6,300,063, 5,874,219, 6,225,625, 5,981,956, 6,141,096, 5,631,734, 6,207,960, 5,925,525, 5,968,740, 6,228,575, 5,837,832, 5,861,242, 6,027,880, 6,309,823, and 6,361,947, which are incorporated herein by reference in their entirety for all purposes.

The use of probe arrays or wafers to decipher genetic information involves the following steps: design and manufacture of probe arrays or wafers, preparation of the sample, hybridization of target nucleic acids to the array, detection of hybridization events and data analysis to determine the sequence or sequences present in the sample. The preferred wafers or probe arrays are manufactured using a process adapted from semiconductor manufacturing to achieve cost effectiveness and high quality, as for example, those manufactured by Affymetrix, Inc.

The design of the wafers or nucleic acid probe arrays begins by probe selection. The probe selection algorithms are based on ability to hybridize to the particular nucleic acid sequence to be scanned. With this information, computer algorithms are used to design photolithographic masks for use in manufacturing the probe arrays.

Probe arrays are preferably manufactured by light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. This parallel process enhances reproducibility and helps achieve economies of scale.

Once fabricated the wafers or nucleic acid probe arrays are ready for hybridization. The nucleic acids to be analyzed (the target) are isolated, optionally amplified and labeled with a fluorescent reporter group. The labeled target is then incubated with the array using a fluidics station and hybridization oven. Optionally, the arrays may be stained following hybridization to facilitate detection of hybridization events. After the hybridization reaction and optional staining is complete, the array is inserted into the scanner, where patterns of hybridization are detected. The hybridization data are collected as light emitted from the fluorescent reporter groups already incorporated into the target, which is now bound to the probe array. Probes most complementary to the target produce stronger signals than those that have mismatches. Since the sequence and position of each probe on the array are known, by complementarity, the identity of the target nucleic acid applied to the probe array can be identified.

It is to be understood that the above description is intended to be illustrative and not restrictive. The scope of the invention should, therefore, be determined not with reference to the above description, but instead with reference to the appended claims along with the full scope of equivalents thereto.

What is claimed is:

1. A method comprising:
(a) identifying one or more genetic variations that at least partly differentiate between a subset of a plurality of individuals having a phenotype-of-interest and a subset of said plurality of individuals not having said phenotype-of-interest;
(b) identifying one or more phenotypes that at least partly differentiate between said subset of said plurality of individuals having said phenotype-of-interest and said subset of said plurality of individuals not having said phenotype-of-interest; and
(c) predicting based upon said one or more genetic variations identified in (a) and said one or more of phenotypes identified in (b), whether a given individual has or does not have said phenotype-of-interest.

2. The method of claim 1 further comprising the step of receiving data on said plurality of genetic variations of said individuals.

3. The method of claim 2 wherein said genetic variations are received from a database.

4. The method of claim 2 wherein said genetic variations are derived by scanning at least 10,000 bases from each of said plurality of individuals.

5. The method of claim 1 wherein said genetic variations are single nucleotide polymorphisms.

6. The method of claim 5 wherein at least one of said single nucleotide polymorphisms is an informative single nucleotide polymorphism.

7. The method of claim 1 further comprising the step of receiving data on a plurality of phenotypes of said individuals.

8. The method of claim 7 wherein said data on said plurality of phenotypes includes data about a degree to which a phenotype of said plurality of phenotypes is present in said individuals.

9. The method of claim 7 wherein said data on said plurality of phenotypes includes data about a degree to which a phenotype of said plurality of phenotypes is absent from said individuals.

10. The method of claim 1 further comprising the step of receiving data on said plurality of genetic variations of said individuals and receiving data on a plurality of phenotypes of said individuals.

11. The method of claim 10 wherein said phenotype-of-interest includes drug response.

12. The method of claim 11 wherein said one or more identified phenotypes and said one or more identified genetic variations at least partly identify one or more individuals from said plurality of individuals for inclusion in a drug trial.

13. The method of claim 11 wherein said one or more identified phenotypes and said one or more identified genetic variations at least partly identify one or more individuals from said plurality of individuals for exclusion from a drug trial.

14. The method of claim 1 wherein said phenotype-of-interest includes disease susceptibility.

15. The method of claim 14 wherein said one or more identified phenotypes and said one or more identified genetic variations at least partly identify one or more individuals from said plurality of individuals for inclusion in a drug therapy.

16. The method of claim 14 wherein said one or more identified phenotypes and said one or more identified genetic variations at least partly identify one or more individuals from said plurality of individuals for exclusion from a drug therapy.

17. The method of claim 10 further comprising the step of scanning at least 10,000 nucleotide bases of a plurality of individuals with and without said phenotype-of-interest.

18. The method of claim 17 wherein said scanning step identifies at least one genetic variation from said plurality of genetic variations.

19. The method of claim 18 wherein said genetic variation has a minor allele frequency of at least 0.1.

20. The method of claim 17 wherein said scanning step includes scanning at least 20,000 bases.

21. The method of claim 17 wherein said scanning step includes scanning at least 50,000 bases.

22. The method of claim 17 wherein said scanning step includes scanning at least 100,000 bases.

23. The method of claim 17 wherein said scanning step includes scanning at least 200,000 bases.

24. The method of claim 17 wherein said scanning step includes scanning at least 500,000 bases.

25. The method of claim 17 wherein said scanning step includes scanning at least 1,000,000 bases.

26. The method of claim 17 wherein said scanning step includes scanning at least 2,000,000 bases.

27. The method of claim 17 wherein said scanning step includes scanning at least 5,000,000 bases.

28. The method of claim 17 wherein said scanning step includes scanning at least 10,000,000 bases.

29. The method of claim 17 wherein said scanning step includes scanning at least 20,000,000 bases.

30. The method of claim 17 wherein said scanning step includes scanning at least 50,000,000 bases.

31. The method of claim 17 wherein said scanning step includes scanning at least 100,000,000 bases.

32. The method of claim 17 wherein said scanning step includes scanning at least 200,000,000 bases.

33. The method of claim 17 wherein said scanning step includes scanning at least 500,000,000 bases.

34. The method of claim 17 wherein said scanning step includes scanning at least 1,000,000,000 bases.

35. The method of claim 17 wherein said scanning step includes scanning at least 2,000,000,000 bases.

36. The method of claim 17 wherein said scanning step includes scanning at least 3,000,000,000 bases.

37. A method comprising
(a) receiving data on a plurality of single nucleotide polymorphisms for a plurality of individuals and data on a plurality of phenotypes for the plurality of individuals; and
(b) using the data on the plurality of single nucleotide polymorphisms and the data on plurality of phenotypes in an association study with a phenotype-of-interest possessed by at least some individuals of the plurality of individuals.

38. The method of claim 37 further comprising the step of predicting whether one or more individuals of the plurality of individuals have or do not have the phenotype-of-interest, based at least on the data on the plurality of single nucleotide polymorphisms and the data on the plurality of phenotypes.

39. A method comprising:
(a) receiving data from an association study between:
(i) a plurality of single nucleotide polymorphisms for a plurality of individuals and data on a plurality of phenotypes for the plurality of individuals, and
(ii) a phenotype-of-interest possessed by at least some of the plurality of individuals; and
(b) predicting whether one or more individuals of the plurality of individuals have or do not have the phenotype-of-interest, based at least on the data from the association study.
